(19) United States
(12) Patent Application Publication
(10) Pub. No.: US 2016/0060285 A1
(43) Pub. Date: Mar. 3, 2016

(54) Aminoglycosides, Methods of Synthesis, and Associated Applications

(71) Applicants: Cheng-Wei Chang, Logan, UT (US); Jon Y. Takemoto, North Logan, UT (US)

(72) Inventors: Cheng-Wei Chang, Logan, UT (US); Jon Y. Takemoto, North Logan, UT (US)

(73) Assignee: UTAH STATE UNIVERSITY, North Logan, UT (US)

(21) Appl. No.: 14/937,681

(22) Filed: Nov. 10, 2015

Related U.S. Application Data
(63) Continuation-in-part of application No. 14/281,659, filed on May 19, 2014.
Application No. 14/281,659 is a continuation-in-part of application No. 13/316,720, filed on Dec. 12, 2011, now Pat. No. 8,865,665.
(60) Provisional application No. 62/077,432, filed on Nov. 10, 2014.
(60) Provisional application No. 61/824,847, filed on May 17, 2013.
(60) Provisional application No. 61/422,983, filed on Dec. 14, 2010.

(51) Int. Cl. C07H 15/234

(52) U.S. Cl. CPC C07H 15/234

(57) ABSTRACT

A fungicidal compound that includes an aminoglycoside, or salt thereof, having the formula:

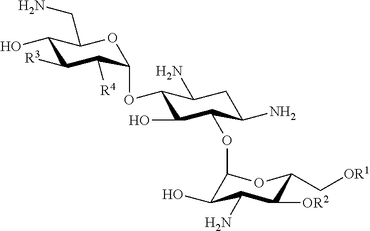

where R¹ is a H; a C₁ to C₂₀ branched or straight alkyl; an unsubstituted phenyl; a substituted phenyl, wherein phenyl group may be C₁ to C₆ alkyl substituted; or a polyethylene glycol —(CH₂CH₂O)ₙR⁵— unit, wherein n=1 to 10 and R⁵═H or C₁ to C₂₀ branched or straight alkyl; R² is a H; a C₁ to C₂₀ branched or straight alkyl; an unsubstituted phenyl; a substituted phenyl, wherein phenyl group may be C₁ to C₁₀ alkyl substituted; or a polyethylene glycol —(CH₂CH₂O)ₙR⁵— unit, wherein n=1 to 10 and R⁵═H or C₁ to C₂₀ branched or straight alkyl; R³ is H or OH; and R⁴ is OH or NH₂.

### RELATED APPLICATIONS

This application (i) claims priority to U.S. Provisional Patent Application No. 62/077,432, filed Nov. 11, 2014 and (ii) is a continuation-in-part to U.S. patent application Ser. No. 14/281,659, filed May 19, 2014 (which in turn claims priority to U.S. Provisional Application No. 61/824,847, filed May 17, 2013 and is a continuation-in-part application to U.S. patent application Ser. No. 13/316,702, filed Dec. 12, 2011, which claims the benefit of U.S. Provisional Application No. 61/422,983, filed Dec. 14, 2010). All of the above referenced applications are incorporated by reference in their entirety.

This application is related to U.S. Pat. No. 8,865,665, issued on Oct. 21, 2014, which is hereby incorporated by reference in their entirety.

### TECHNICAL FIELD

The present disclosure relates to antimicrobials. More particularly, this disclosure relates to antifungal aminoglycosides, their synthesis, and various applications.

### BACKGROUND

Aminoglycoside antibiotics have been commonly used as a medical treatment against infectious diseases for over 60 years, although the prevalence of aminoglycoside resistant bacteria has significantly reduced their effectiveness. Aminoglycosides have two or more amino sugars bound to an aminocyclitol ring through glycosidic bonds. Naturally occurring aminoglycosides (produced by Actinomycetes) are widely used as antibiotics against bacterial infections of animals and humans. These include the well-known antibiotics kanamycin, streptomycin, and neomycin. Aminoglycoside antibiotics are believed to act on the bacterial protein synthesis machinery, leading to the formation of defective cell proteins.

Kanamycin is a known aminoglycoside antibiotic. The antibiotic function of kanamycin may be related to its ability to affect the 30S ribosomal subunit of bacteria, causing frameshift mutations or preventing the translation of RNA. Either frameshift mutations or a lack of RNA translation can lead to a reduction or absence of bacterial protein synthesis and, ultimately, to bacterial death. Unfortunately, kanamycin has been rendered all but obsolete for clinical use due to the emergence of resistant bacteria.

In medicine, fungal diseases have emerged over the last 25 years as a major public health problem. Among the prominent reasons for this increase is the lack of efficacious antifungal agents, increases in immunocompromised conditions (e.g., organ transplants and HIV/AIDS), and widespread resistance to the most commonly used antifungals. The strongest medically used antifungal agent, amphotericin B, is an effective medication, but is also highly toxic to patients. The toxicity levels of the available antifungal medications are a common concern for medical practitioners. U.S. Pat. No. 5,039,666 to Novick, Jr. (1991) shows an aminoglycoside compound “gentamicin” having reduced nephrotoxicity induced by the aminoglycoside. Other common antifungal medications are used to treat infections such as athlete's foot, ringworm, candidiasis (thrush) and serious systemic infections such as cryptococcal meningitis, and others.

In agriculture, the control of crop diseases by direct application of biocides remains the most effective and most widely used strategy. Nevertheless, concerns with inconsistent and declining effectiveness, environmental impacts, animal/human toxicity, and costs continue to challenge the use of existing biocides. For example, the crop pathogen Fusarium graminearum, the most common causative agent of head blight disease in wheat and barley in North America, is difficult to predict and can result in catastrophic crop loss.

There exists a need for novel antimicrobials to address the problems of resistant bacteria and fungi, both in human medicine and in crop disease. There is also a clear need for novel antimicrobials, especially antifungals, with reduced toxicity. Furthermore, it would be desirable for new antimicrobial compounds to be selective against either bacteria or fungi, so treatment for one of either bacterial or fungal disease does not contribute to the buildup of antimicrobial resistance in the other. Selective antimicrobial activity is especially desirable for antifungals used to treat crop disease, such as Fusarium head blight, due to the possibly large amounts of antimicrobial agent released into the environment when crops are treated.

U.S. Pat. No. 8,865,665, issued on Oct. 21, 2014 and sharing common inventors, discloses a series of fungicidally effective aminoglycosides. In U.S. patent application Ser. No. 14/281,659, filed on May 19, 2014 and also sharing common inventors, a series of aminoglycosides are combined with azoles, resulting in synergistic fungicidal effects. Even with new compounds, combinations, and associated methods disclosed in the above patent and patent application, additional, novel fungicides are needed. The present invention provides for such additional novel aminoglycoside antimicrobials that are effective, have relatively low levels of toxicity, and are selective against fungal pathogens.

### SUMMARY

The present disclosure in aspects and embodiments addresses these various needs and problems by providing a fungicidal compound comprising:

an aminoglycoside, or salt thereof, having the formula:

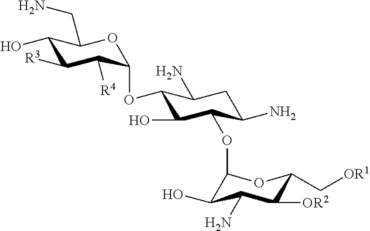

wherein:
R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit,
   wherein n=1 to 10 and R⁵═H or C₁ to C₂₀ branched or straight alkyl;
R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit,
   wherein n=1 to 10 and R⁵═H or C₁ to C₂₀ branched or straight alkyl;
R³ is a member selected from the group consisting of H and OH; and
R⁴ is a member selected from the group consisting of OH and NH₂.

In addition, the present disclosure provides methods of synthesizing the fungicidal compounds, methods of administering the fungicidal compounds, and fungicidal compounds that combine the above compounds with azoles.

### DETAILED DESCRIPTION

The present disclosure covers compositions, kits, reagents, and associated methods for microbial aminoglycoside compounds. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

### A. DEFINITIONS

Before discussing the present invention in further details, the following terms, when and if used, and conventions will first be defined:

Host: The term “host” is defined herein as any living organism infected or at least somewhat likely of being infected by a fungal pathogen, where said pathogen and any infection caused by said pathogen, or potential infection caused by said pathogen, are susceptible to treatment with one or more of the compounds of Formula I as claimed herein, where said treatment is likely to result in the elimination, avoidance, or alleviation of the infection caused by said pathogen.

Kanamycin A: For comparative purposes “Kanamycin A” has the following structure:

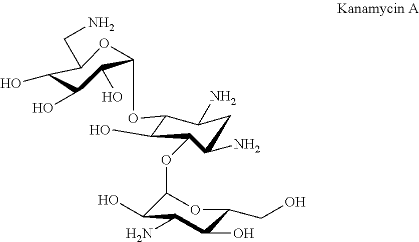

Kanamycin B: For comparative purposes “Kanamycin B” has the following structure:

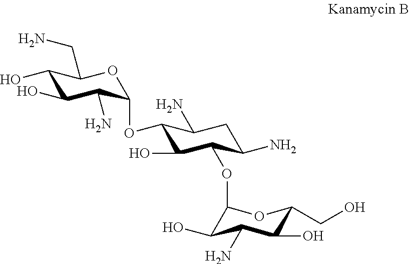

Tobramycin: For comparative purposes “Tobramycin” has the following structure:

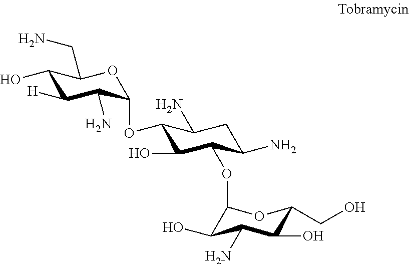

Fungal Infection: The term “fungal infection” is defined herein as an association of a fungal organism with a host, whether said association is actual or potential. For example, an actual associate occurs when a fungus is physically present on or within a host. Examples of potential associations include fungi on or within the environment surrounding a host, where a fungus is at least somewhat likely to be actively or passively transferred to the host. Without wishing to further limit the type of associations between a fungal organism and host, examples of the association of the fungal organism with the host include biological associations that may be pathogenic or non-pathogenic, parasitic or non-parasitic, symbiotic or non-symbiotic, mutualistic or non-mutualistic, commensal, naturally occurring or man-made, or any other biological interaction.

Host in need thereof: The phrase “host in need thereof” is defined herein as any host associated or potentially associated with a fungal organism, where said host may actually or potentially benefit from elimination, prevention, or alleviation of a fungal infection.

Fusarium Head Blight: The phrase “fusarium head blight” is defined herein as any fungal disease caused by the fungus Fusarium graminearum.

Surfactant: The term “surfactant” is used to indicate the common laboratory surfactant C₅₈H₁₁₄O₂₆. All uses of the term “surfactant” refer to C₅₈H₁₁₄O₂₆, unless otherwise indicated.

Prophylactically: The term “prophylactically” is used herein to refer to the administration of an antimicrobial compound for the prevention of disease.

N/A: As used herein to describe data points, the abbreviation “N/A” means not tested.

Adjuvant: The term “adjuvant” is defined herein as a substance that helps and enhances the pharmacological effect of a drug or increases the ability of an antigen to stimulate the immune system.

Excipient: The term “excipient” is defined herein as an inactive substance used as a carrier for the active ingredients of a medication.

Diluent: The term “diluent” is defined herein as any liquid or solid material used to dilute or carry an active ingredient.

Antifungal Amount or antifungal effective: Unless otherwise specified, the phrases “antifungal amount” or “Antifungal Effective” are used herein to describe an amount of an antifungal agent sufficient to reduce, eliminate, or alleviate a fungal infection or the symptoms of a fungal infection on or within a host.

MIC: The term MIC means the “minimal inhibitory concentration” or the lowest concentration of a test compound observed to inhibit the visible growth of the test microbe after 24 or 48 hours of incubation.

MFC: The term MFC means the “minimum fungicidal concentration” or the lowest concentration of the test compound that shows either no growth or fewer than three growing colonies of a fungus on an agar growth medium surface after 48 hours of incubation.

Admixed: The term “admixed” is used herein to describe a chemical or compound in a mixture or combination with other chemicals or compounds.

Administering: The term “administering” is defined herein to describe the act of providing, exposing, treating, or in any way physically supplying or applying a chemical or compound to any living organism or inanimate object associated with a living organism, where said organism will actually or potentially benefit for exposure, treatment, supplying or applying of said chemical or compound.

Topical: The term “topical” is defined herein as pertaining to the surface of a body part, surface part of a plant, or surface of an inanimate object or composition, such as soil. For example, in medicine, a topical medication is applied to body surfaces such as the skin or mucous membranes, for example throat, eyes and ears.

Carrier: The term “carrier” is defined herein as any substance that serves to improve the delivery and the effectiveness of a drug or antimicrobial agent and is inclusive of excipients as defined above. Examples include: microspheres made of biodegradable polymer poly(lactic-co-glycolic) acid, albumin microspheres, synthetic polymers (soluble), protein-DNA complexes, protein conjugates, erythrocytes, nanoparticles, and liposomes

Grain head: The phrase “grain head” as used herein is meant to include both small and large grains.

Warm-blooded animal: Used herein the phrase “warm-blooded animal” means an animal characterized by the maintaining of a relatively constant and warm body temperature independent of environmental temperature; homeothermic.

Certain terms in this application are to be interpreted as commonly used in the technical fields of medicine, antimicrobials, and crop disease, as indicated by the context of their use. These terms include spray nozzle, droplet, therapeutically, exterior, spraying, topical, treatment, and prevention.

PDB-CA: The term PDB-CA refers to potato dextrose broth+casamino acids medium used for fungal growth and as diluent in MIC tests. To make 1 L of PDB-CA, 200 g of diced fresh potatoes were boiled in 500 mL of distilled water for 30 min. The broth was filtered through 2 layers of cheesecloth, and the volume was brought up to 1 L. After addition of 20 g of glucose (2%, w/v) and 4 g of casamino acid (0.4%, w/v), the mixture was stirred with a magnetic bar until all solids were dissolved. The pH was adjusted with HCl and NaOH to 5.1. Then, the medium was sterilized by autoclaving for 30 min.

PDA-CA: The term PDA-CA refers to a solid growth medium composed of 2% agar dissolved in PDB-CA. The mixture was sterilized by autoclaving for 30 min, poured into sterile Petri dish plates, and solidified in the plates upon cooling to room temperature.

RPMI 1640: The term RPMI 1640 refers to a chemically defined cell growth medium composed of twenty amino acids, eleven vitamins, calcium nitrate (Ca(NO₃)₂4H₂O), magnesium sulfate (MgSO₄) (anhyd.), potassium chloride (KCl), sodium chloride (NaCl), sodium phosphate dibasic (Na₂HPO₄) anhydrous, dextrose, glutathione (reduced) and buffered to a pH of 7.0 with 0.165 M morpholinepropanesulfonic acid (MOPS) buffer (as described by Moore, G. E., Gerner, R. E. and Franklin, H. A. (1967) J. Amer. Med. Assoc. 199:519). For the studies described herein RPMI 1640 was purchased from Sigma-Aldrich Chemical Co., St. Louis, Mo., USA).

### B. AMINOGLYCOSIDES

The present disclosure relates to antimicrobial aminoglycosides. Exemplary aminoglycosides include the compound of Formula I as follows:

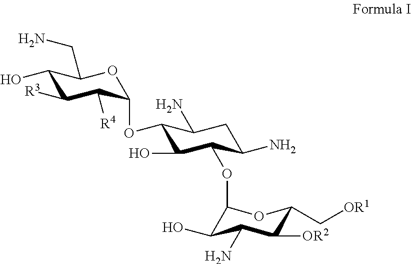

wherein:

R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit, wherein n=1 to 10 and R⁵═H or C₁ to C₂₀ branched or straight alkyl;

R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit, wherein n=1 to 10 and R⁵═H or C₁ to C₂₀ branched or straight alkyl;

R³ is a member selected from the group consisting of H or OH; and

R⁴ is a member sleeted from the group consisting of OH or NH₂.

In embodiments, at least one of R¹ and R² is substituted with a group other than hydrogen.

In some embodiments, when both of R¹ and R² are substituted with a group other than hydrogen, R¹ and R² are substituted with the same group.

In other embodiments, when both of R¹ and R² are substituted with a group other than hydrogen, R¹ and R² are substituted with different groups.

In some embodiments, Formula I may be adjusted to replace one or both of the O next to R¹ and R² with an S.

In some embodiments, R¹ and R² are substituted with a crown ether, as set forth in the synthetic approaches illustrated below.

In some embodiments, R¹ and R² may be selected from the group consisting of C(O)OR³ (alkoxycarbonyl), C(O)NHR³ (alkylaminocarbonyl), S(O)₂R³ (alkylsulfonyl), S(O)₂R⁴ (phenylsulfonyl), S(O)R³ (alkylsulfinyl), P(O)₂R³ (alkylphosphonyl), and C(O)R³ (alkanoyl).

In the carbon chains described above, any integer within the described range may be used. For example, R¹ and R² may be a branched or straight chain C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀ alkyl group.

An exemplary embodiment includes (referred to as K4608):

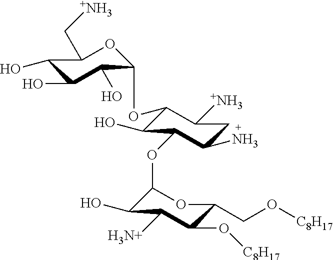

Another exemplary embodiment includes (referred to as K408):

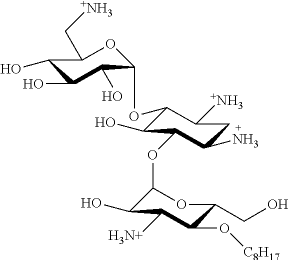

Another exemplary embodiment includes (referred to as K608):

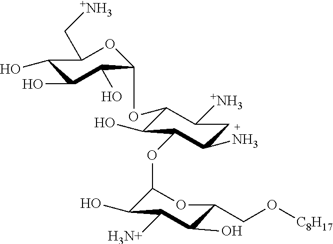

### C. SYNTHESIS

The aminoglycosides disclosed herein may be prepared according to the following general synthetic approaches:

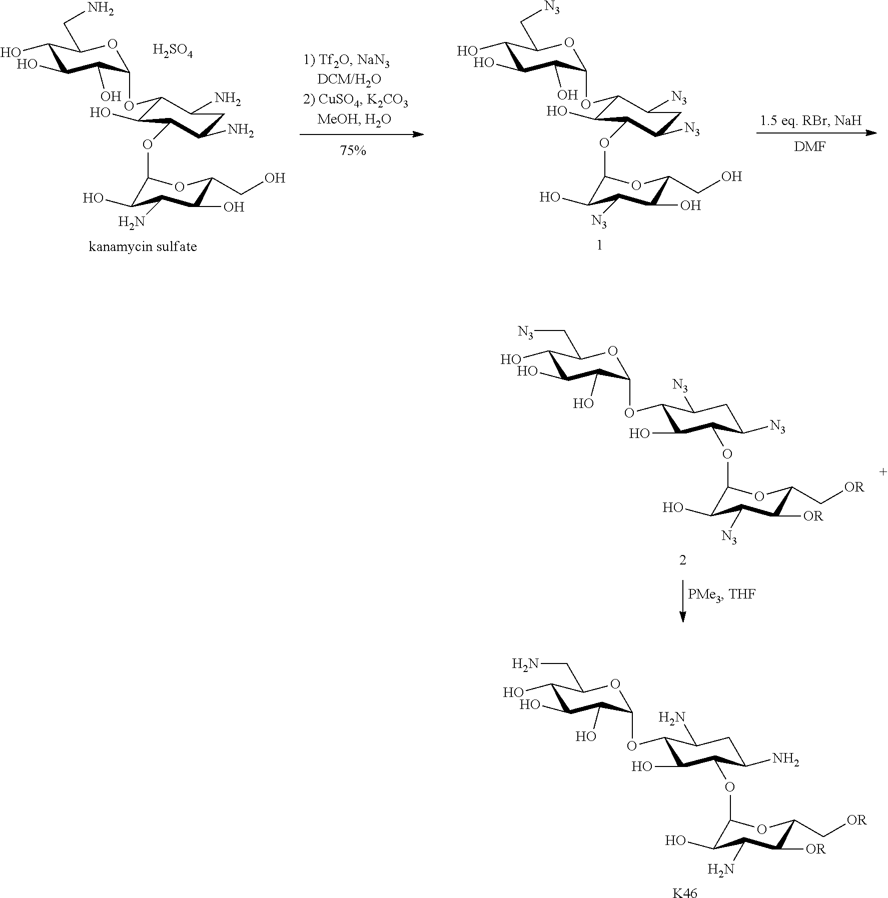

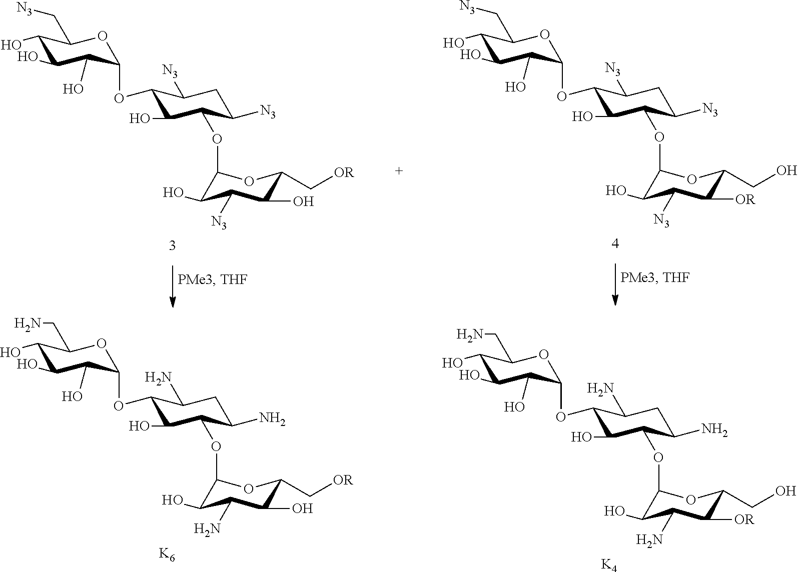

Other suitable synthetic approaches may also be employed.

Related synthetic approaches for synthesizing crown ether moieties include the following approaches:

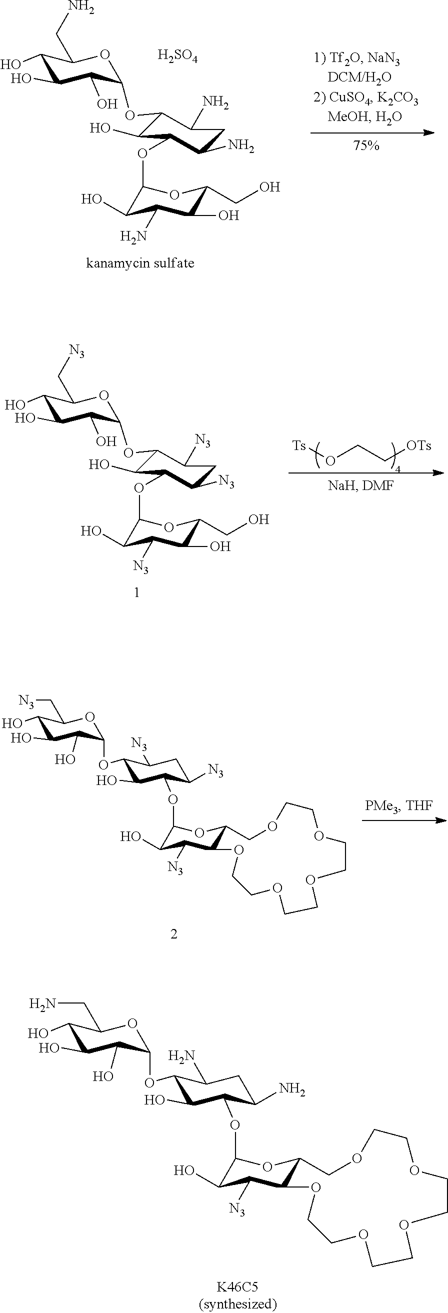

In embodiments, the crown moiety may be varied in size by adjusting the ether component. As shown, for example, below:

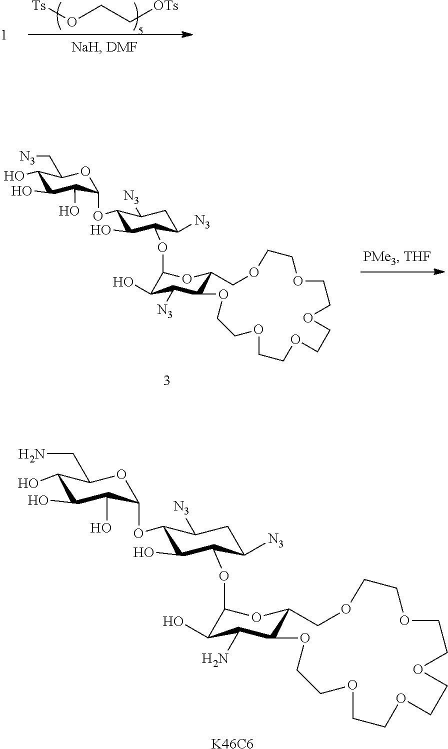

The structure related to the compounds described herein may be derived from a parent aminoglycoside molecule other than kanamycin A, such as kanamycin B and tobramycin, that is capable of being modified by the addition of a variety of substituents on ring III equivalent of the ring III of kanamycin A. Particularly preferred is the addition of a carbon alkyl chain as designated herein on ring III.

In some embodiments, the aminoglycosides may be derived from the parent aminoglycoside molecule by the synthesis method shown herein, but the substituent, such as the carbon alkyl chain on ring III of the structure related to the present invention varies in the number of carbon atoms and hydrogen atoms.

### D. APPLICATIONS

The compounds described herein may be used to treat a variety of fungal infections in a large variety of hosts. Exemplary fungi that may be treated with the aminoglycosides include: Fusarium graminearum, Fusarium oxysporum, Fusarium culmorum, Aspergillus flavus, Aspergillus fumigatus, Aspergillus niger, Altenaria solani, Botrytis cinerea, Botrytis alcata, Phakapsora pachyrhizi, Candida albicans, Candida pseudotropicalis, Candida rugosa, Candida parapsilosis, Candida lusitaniae, Cryptococcus gattis and Cryptococcus neoformans. One preferred embodiment of the present invention is the treatment of fungal infection in a host in need thereof, where the elimination or reduction of bacteria associated with said host is undesirable. Without wishing to limit the scope of the invention in any way, one such use could occur in human or non-human mammals, where treatment of a fungal infection with and aminoglycoside would inhibit, eliminate, or otherwise alleviate the fungal infection, but not affect the integrity of the intestinal flora of the host. Again, without limiting the invention, a second example is the treatment or prevention of fungal disease in a host crop, where it is undesirable to affect the diversity or abundance of bacteria of said host crop. In some embodiments, the novel antifungal compounds may be used to treat the soil or plants to eliminate fungal growth and activity.

In still yet another broad embodiment, the present invention is used to treat a variety of fungal pathogens related to human, crop, or animal disease.

In further embodiments, the compound of the present invention is administered by spraying, direct injection, topical application, ingestion (including pharmaceutical compositions that include the structure related to the present invention), or by inclusion in the water supply, to either a human, an animal, or a crop immediately threatened by, or potentially threatened by, a fungal pathogen, where fungal pathogen is causing or may cause fungal disease, and administration of the compounds of the present invention will reduce, eliminate, or avoid fungal disease.

### D. AZOLES AND COMBINATIONS

The aminoglycosides as described above may be combined with one or more of suitable azoles. Azoles include compounds having a five-membered nitrogen heterocyclic ring containing at least one other non-carbon atom of either nitrogen, sulfur, or oxygen, such as azoles that include 1 nitrogen atom, 2 or more nitrogen atoms, 1 nitrogen atom and 1 oxygen atom, and 1 nitrogen atom and 1 sulfur atom. The five-membered nitrogen heterocyclic ring may be additionally substituted.

In some embodiments where the compounds are employed for biocidal or antibiotic effects, suitable azoles include those that have at least some desired biocidal effect. Exemplary azoles include pyrroles, pyrazoles, imidazoles, triazoles, tetrazoles, pentazoles, oxazoles, isoxazoles, thiazoles, and isothiazoles.

In some embodiments, the azole may be selected from one or more of the following: itraconazole, fluconazole, voriconazole, posaconazole, chlotrimazole, tioconazole, ketocaonazole, metconazole, tebuconazole, and pyraclostrobin.

In embodiments, the azoles may be used as illustrated above, as salts, or any other suitable form for delivery to a target organism.

The aminoglycosides and azoles may be used in any suitable ratio or combination. The ratio of azole to aminoglycoside may be from about 1:1 to about 1:1000, from about 1:5 to about 1:600, from about 1:20 to about 1:500, from about 1:30 to about 1:200, and from about 1:50 to about 1:100. For example, in some embodiments, the ratio of azole:aminoglycoside may be about 1:5, 1:21, 1:32, 1:53, 1:180, and 1:533.

One preferred embodiment of the present invention is the treatment of fungal infection in a host in need thereof, where the elimination or reduction of bacteria associated with said host is undesirable. Without wishing to limit the scope of the invention in any way, one such use could occur in human or non-human mammals, where treatment of a fungal infection with and aminoglycoside of the invention such as K20 would eliminate or alleviate the fungal infection, but not affect the integrity of the intestinal flora of the host. Again, without limiting the invention, a second example is the treatment or prevention of fungal disease in a host crop, where it is undesirable to affect the diversity or abundance of bacteria of said host crop.

In a broad embodiment, the present invention is drawn to novel antifungal compounds, a method to synthesize said novel antifungal compounds, and methods to use said novel antifungal compounds to treat humans, animals, soil, or plants to eliminate fungal growth and activity. In one broad embodiment, the structure related to the present invention is derived from a parent aminoglycoside molecule other than kanamycin A that is capable of being modified by the addition of a variety of substituents on ring III equivalent of the ring III of kanamycin A. Particularly preferred is the addition of a carbon alkyl chain as designated herein on ring III.

In yet another broad embodiment the present invention is derived from the parent aminoglycoside molecule by the synthesis method shown herein, but the substituent, such as the carbon alkyl chain on ring III of the structure related to the present invention varies in the number of carbon atoms and hydrogen atoms.

In still yet another broad embodiment, the present invention is used to treat a variety of fungal pathogens related to human, crop, or animal disease. In further broad embodiments, the compound of the present invention is administered by spraying, direct injection, topical application, ingestion (including pharmaceutical compositions that include the structure related to the present invention), or by inclusion in the water supply, to either a human, an animal, or a crop immediately threatened by, or potentially threatened by, a fungal pathogen, where fungal pathogen is causing or may cause fungal disease, and administration of the compounds of the present invention will reduce, eliminate, or avoid fungal disease.

### EXAMPLES

The following examples are illustrative only and are not intended to limit the disclosure in any way. The following materials and methods were used in either one or more of the examples listed below. Further materials and methods are provided in the description of each example.

### I. Aminoglycoside Synthesis

All aminoglycosides were provided by the laboratory of Dr. Cheng-Wei T. Chang (Department of Chemistry and Biochemistry, Utah State University). For antifungal tests, stock solutions were prepared as 10 mg mL⁻¹ solutions in water and stored at minus 20° C. Synthesis of the aminoglycosides followed the following procedure.

Step 1. Synthesis of Azidokanamycin, 1:

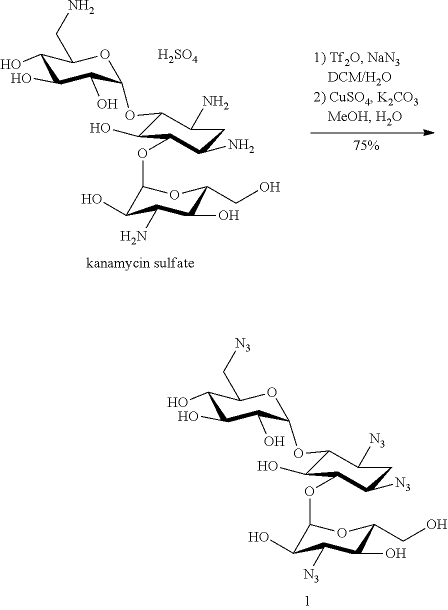

Procedure for Synthesis of Azidokanamyin (1):

To a solution of NaN₃ (25.4 g, 388 mmol) in a mixture of H₂O/CH₂Cl₂ (200 mL, 1:1 v/v) at 0° C., Tf₂O (33 mL, 199 mmol) was added. The reaction mixture was stirred at room temperature for 2 h. After being quenched with saturated aqueous NaHCO₃, the layers were separated, and the aqueous layer was extracted with CH₂Cl₂ (100 mL). The organic layers were combined to afford 200 mL of TfN₃ solution, which was added to a solution of kanamycin sulfate (9.45 g, 16.2 mmol) in H₂O (200 mL), MeOH (600 mL), and CuSO₄ (250 mg) prepared as a stock solution of MeOH/H₂O/Et₃N (50 mL, 3/3/4 v/v/v). The reaction mixture was stirred at room temperature for 2 h. Then solid NaHCO₃ (20 g) was added carefully. After being stirred for 30 min, the reaction mixture was filtered through filter paper suing Buchner funnel. The solvents were removed and EtOAc (500 mL) was added. The solution was filtered again to remove the residual inorganic salts. The filtrate was mixed with silica gel (ca. 20 g) and the solvent was removed by rotovap to give greenish powder. The greenish powder was loaded to a column and purified via a gradient column chromatography (EtOAc:MeOH from 100:0 to 50:50) to afford tetraazidokanamycin, 1 in 60-85% yield.

Step 2. Alkylation of Azidokanamycin:

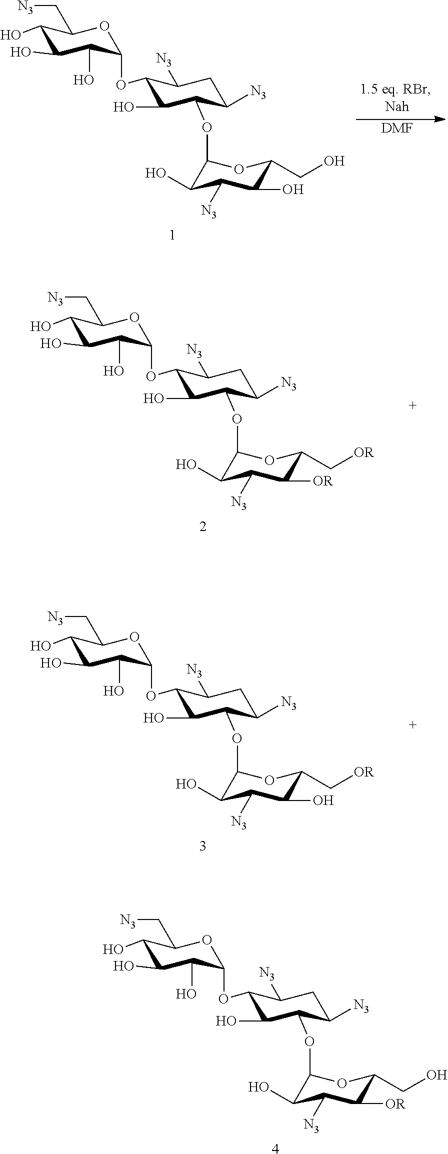

General Procedure for O-Alkylation of Azido Kanamycin Derivatives (2, 3 and 4):

To a solution of azidokanamycin (0.5 g, 0.85 mmol) in 20 mL of anhydrous DMF, NaH (20 equiv.) was added. The reaction mixture was stirred for 10 minutes before adding the corresponding alkyl bromide (1.5 equiv.). The reaction mixture was stirred at room temperature overnight. Completion of the reaction was confirmed by TLC (Eluted with EtOAc), and the reaction was quenched by adding MeOH (0.5 mL). The mixture was concentrated and purified with a gradient column chromatography (eluted from EtOAc:Hexane 30:70 to 100:0) to obtain three products 2, 3 and 4 (in order of eluting out from the column).

Step 3. Reduction of Azido Groups:

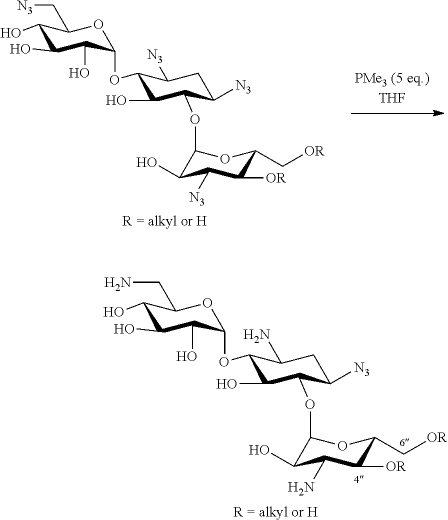

General Procedure for Staudinger Reaction:

the O-alkylated azidokanamycin (0.2 g) was dissolved into 20 mL THF in a 50 mL round bottom flask. Several drops of water and 5 equiv. of PMe₃ (1 M solution in THF) was added to the solution. The reaction mixture was heated at 60° C. for 2 hours. The reaction mixture was concentrated and then diluted with water. After being filtered through Celite, the crude product was loaded to a CG50 column (NH₄⁺ form) and purified with a gradient elution of water and ammonium hydroxide (H₂O:NH₂OH from 100:0 to 50:50) to afford the desired product. The product was concentrated to dryness and then acidified with 5% HOAc in water to afford the acetate salt in water. The acetate salt can be exchanged through an ion-exchange column (Dowex 1×8 chloride form) to afford the final product as a chloride salt.

Results. Isolated Yields for Steps 2 and 3:

Isolated Yields (%) from Step 2:

| R | 2 | 3 | 4 |
|---|---|---|---|
| C₄H₉ | 22 | 3^{a} | 22 |
| C₆H₁₃ | 28 | 3^{a} | 21 |
| C₇H₁₅ | 30 | 7^{a} | 36 |
| C₈H₁₇ | 25 | 15 | 26 |
| C₁₂H₂₅ | 21 | 13 | 28 |
| C₁₀H₂₁ | 30 | 7 | 24 |
| C₉H₁₉ | 34 | 13 | 25 |
| C₁₄H₂₉ | 21 | 4^{a} | 16 |
| C₁₆H₃₃ | 19 | 5^{a} | 25 |

^{a}estimated from 1H NMR

Isolated Yields (%) from Step 3:

| Compounds | Yields (%) | Type and Position of R Group |
|---|---|---|
| K4604 | 71% | Butyl (C4) at 4″ and 6″ |
| K604 | Not isolated | Butyl (C4) at 6″ |
| K404 | 17% | Butyl (C4) at 4″ |
| K4606 | 38% | Hexyl (C6) at 4″ and 6″ |
| K606 | Not isolated | Hexyl (C6) at 6″ |
| K406 | 67% | Hexyl (C6) at 4″ |
| K4607 | 89% | Heptyl (C7) at 4″ and 6″ |
| K607 | Not isolated | Heptyl (C7) at 6″ |
| K407 | 46% | Heptyl (C7) at 4″ |
| K4608 | 82% | Octyl (C8) at 4″ and 6″ |
| K608 | 47% | Octyl (C8) at 6″ |
| K408 | 91% | Octyl (C8) at 4″ |
| K4609 | 84% | Nonyl (C9) at 4″ and 6″ |
| K609 | 19% | Nonyl (C9) at 6″ |
| K409 | 20% | Nonyl (C9) at 4″ |
| K4610 | 59% | Decyl (C10) at 4″ and 6″ |
| K610 | 66% | Decyl (C10) at 6″ |
| K410 | 21% | Decyl (C10) at 4″ |
| K4612 | 62% | Dodecyl (C12) at 4″ and 6″ |
| K612 | 52% | Dodecyl (C12) at 6″ |
| K412 | 38% | Dodecyl (C12) at 4″ |
| K4614 | 76% | Tetradecyl (C14) at 4″ and 6″ |
| K614 | Not isolated | Tetradecyl (C14) at 6″ |
| K414 | 60% | Tetradecyl (C14) at 4″ |
| K4616 | 65% | Hexdecyl (C16) at 4″ and 6″ |
| K616 | Not isolated | Hexdecyl (C16) at 6″ |
| K416 | 31% | Hexdecyl (C16) at 4″ |

### II. Bacterial and Fungal Growth Inhibitory Tests

To test the efficacy of the above prepared compounds, the following procedures were used.

1. Microbe Source and Methods for Maintenance and Culture

Organisms and culture conditions. Fusarium graminearum B4-5A was obtained from the Small Grain Pathology Program, University of Minnesota, Minneapolis Minn., USA. Escherichia coli ATCC 25922, Staphylococcus aureus ATCC25923, Candida albicans ATCC64124 (azole-resistant) and Candida albicans ATCC MYA-2876 (azole sensitive) were obtained from the American Type Culture Collection (Manassas, Va., USA). Fusarium graminearum B4-5A and Candida albicans strains were maintained on potato dextrose agar (PDA) and cultivated at 28° C. in potato dextrose broth (PDB) or at 35° C. with RPMI 1640 (with L-glutamine, without sodium bicarbonate (Sigma-Aldrich Chemical Co., St. Louis, Mo., USA) buffered to pH 7.0 with 0.165 M morpholinepropanesulfonic acid. For maintenance and culture inocula E. coli ATCC 25922 was grown at 37° C. for 24 h on Luria-Bertani (LB) medium (See Sambrook, J., Fritsch, E. F., and Maniatis T. (1989), Molecular cloning: a laboratory manual, Cold Spring Harbor Laboratory Press, Cold Spring Harbor, N.Y., USA) and S. aureus ATCC 25922 was grown on Mueller-Hinton medium (Difco, BD, Franklin Lakes, N.J., USA). In embodiments any suitable growth media may be used.

2. Bacterial and Fungal Growth Inhibitory Tests

Bacterial Growth Inhibition (MIC) Tests.

In vitro effects of the aminoglycosides on the growth of Escherichia coli ATCC 25922 and Staphylococcus aureus ATCC25923 were assayed in 96-well uncoated polystyrene microtiter plates and minimal inhibitory concentration (MIC) values were determined using Clinical and Laboratory Standards Institute (CLSI) standard protocols. See National Committee for Clinical Laboratory Standards (1993), Methods for Dilution Antimicrobial Susceptibility Tests for Bacteria that Grow Aerobically—Third Edition: Approved Standard M7-A3. Villanova, Pa.: National Committee for Clinical Laboratory Standards. Bacterial cells were grown overnight in Luria-Bertani medium (See Sambrook et al., 1989), and diluted to a concentration of 1×10⁴ CFU/mL. Ten aliquots of the diluted overnight culture were then added to 190 μL of Luria-Bertani medium dispensed in the microtiter plate wells each containing a specific aminoglycoside at concentrations ranging between 4 and 500 mg/L. Controls were bacterial cells only with no aminoglycoside added to separate wells. The plates were incubated at 37° C. without shaking for 24 hours before determination of MIC values. Controls were no cells and no aminoglycoside added to separate wells. MIC was defined as the “minimal inhibitory concentration” meaning the lowest concentration of a test compound observed to inhibit visible growth of the test microbe after 24 or 48 hours of incubation. Experiments were performed in triplicate.

Fungal Minimal Inhibitory Concentration (MIC) and Minimal Fungicidal Concentration (MFC) Tests.

In vitro effects of aminoglycosides on the growth of fungal yeast strains Candida albicans ATCC64124 (azole-resistant) and Candida albicans ATCC MYA-2876 (azole sensitive) were determined using microbroth dilution assays in 96-well uncoated polystyrene microtiter plates (Corning Costar, Corning, N.Y., USA) as described in the M27-A3 reference methods of the Clinical and Laboratory Standards Institute with minor modifications. See National Committee for Clinical Laboratory Standards (2002). Reference Method for Broth Dilution Antifungal Susceptibility Testing of Yeasts—Third Edition: Approved Standard M27-A3. Wayne, Pa.: National Committee for Clinical Laboratory Standards. Modifications included growing cell inocula in RPMI 1640 medium (Catalog #11875 Life Technologies, San Diego, Calif. USA) for 48 hours at 35° C. and suspending fresh-grown inocula to a concentration of 5×10⁴ cells/mL (determined by hemocytometer cell counting) in fresh RPMI 1640 for the assays. All yeast cell suspensions (100 μL) containing 0.49 to 500 mg/L of serial diluted aminoglycoside were added to the wells of a 96-well microtiter plate and incubated for 48 hours at 35° C. Controls were no cells and no aminoglycoside added to separate wells. Each test was performed in triplicate.

For in vitro antifungal activities against F. graminearum B4-5A, spores were prepared as described previously. See Lay F. T., Brugliera, F. and Anderson, M. A. (2003). Isolation and Properties of Floral Defensins from Ornamental Tobacco and Petunia. Plant Physiology. 131, 1283-1293. Spores were isolated from sporulating cultures growing in potato dextrose broth (PDB) medium by filtration through sterile glass wool. Microbroth dilution assays for determination of MICs were conducted using the M38-A2 protocols of the Clinical and Laboratory Standards Institute with minor modification. See National Committee for Clinical Laboratory Standards (2008). Reference Method for Broth Dilution Antifungal Susceptibility Testing of Filamentous Fungi—Second Edition: Approved Standard M38-A2. Wayne, Pa.: National Committee for Clinical Laboratory Standards. Serial dilutions of K20 were made in uncoated polystyrene 96-well plates in the range of 0.48 to 250 mg/L using RPMI 1640 medium and spore suspensions were added to make a final concentration of 5×10⁵ CFU/mL. The plates were incubated at 35° C. for 48 hour. Each test was performed in triplicate.

MFC tests were performed after 48 hours of incubation and evaluation of MIC test plates. See Espinel-Ingroff, A., et al. (2002). Testing Conditions for Determination of Minimum Fungicidal Concentrations of New and Established Antifungal Agents for Aspergillus spp.: NCCLS Collaborative Study. Journal of Clinical Microbiology 40(9): 3204-3208. Without agitating the plates, 10 μL of the liquid contents from the MIC microtiter plate wells that showed complete inhibition (100% or an optically clear well) before the first positive well (showing growth similar to that for the growth control well) was streaked out (using a sterile wire loop) or spread-plated onto Sabouraud dextrose agar (Becton, Dickinson and Company, Sparks, Md. USA) plates. As a control, growing cells from the positive growth-control well were also streaked-out or spread-plated onto Sabouraud dextrose agar plates. The agar plates were incubated at 35° C. until growth was seen in the growth control subculture (usually before 48 hour). The MFC values were determined as the lowest aminoglycoside concentrations that shows either no growth or fewer than three growing colonies on agar surfaces reflecting approximately 99 to 99.5% killing activity.

Results.

Results are shown in the following tables.

Minimal inhibitory concentrations of aminoglycosides against Fusarium graminearum B4-A5, Escherichia coli ATCC 25922, and Staphylococcus aureus ATCC25923:

| | MIC (mg per L) | | |
|---|---|---|---|
| | E. coli | S. aureus | F. graminearum |
| Compound | 25922 | 25923 | B4-5A |
| K404 | 125->250 | 4-16 | >500 |
| K406 | >250 | 8-64 | >500 |
| K407 | >250 | 125 | 125 |
| K408 | 125->250 | 125 | 31.3 |
| K409 | >250 | >250 | 62.5 |
| K410 | >250 | 125 | 62.5 |
| K412 | 125-250 | 64 | >500 |
| K414 | >250 | >250 | >500 |
| K4604 | >250 | >250 | 125 |
| K4606 | >250 | 32-125 | 3.9 |
| K4607 | 125 | 8-125 | 15.6 |
| K4608 | >250 | >250 | 15.6 |
| K4609 | 16 | 8 | 125 |
| K4610 | 125 | 16 | >500 |
| K4612 | >250 | 64 | >500 |
| K4614 | >250 | >250 | >500 |
| Kanamycin | 1-4 | 0.5-1 | >500 |

Minimal inhibitory concentrations (MICs) and minimal fungicidal concentrations (MFCs) of aminoglycosides against Candida albicans strains 64124 (azole-resistant) and MYA 2876 (azole sensitive)

| MIC or MFC (mg per L) | |
|---|---|
| | C. albicans |
| C. albicans 64124 | MYA2876 |

| Compound | MIC | MFC | MIC | MFC |
|---|---|---|---|---|
| K408 | 9.8 | 9.8 | 1.2 | 2.4 |
| K4608 | 4.9-9.8 | 9.8 | 1.2-2.4 | 2.4 |
| kanamycin | >500 | not tested | 250-500 | not tested |

### III. Synergistic Antifungal K-Compound and Azole Combinations

To test the efficacy of the above prepared compounds in combination with azoles, the following procedures were used.

Test Procedures

Antifungal synergism was determined by checkerboard analysis (Clinical and Laboratory Standards Institute. 2008. Reference Method for Broth Dilution Antifungal Susceptibility Testing of Yeasts—Approved Standard. CLSI document M27-A3. Clinical and Laboratory Standards Institute, Wayne, Pa.). Final concentrations of yeast cells or fungal spores was adjusted to a viable cell density of 1×10⁵ CFU/mL as determined by colony counting on agar plates. K-compound and antifungal azole drug concentration ranges optimal for checkerboard analyses were employed. The concentration ranges were 0.015 to 50 mg L⁻¹ for itraconazole; 0.12 to 250 mg L⁻¹ for fluconazole; 0.015 to 250 mg L⁻¹ for voriconazole; 0.03 to 25 mg L⁻¹ for chlotrimazole; 0.06 to 12.5 mg L⁻¹ for posaconanzole; and 0.25 to 62.5 mg L⁻¹ for K-compound. A fractional inhibitory concentration (FIC) non-parametric model based on Loewe additivity theory was used (Meletiadis J, Mouton J W, Meis J F, Verweij P E. In vitro drug interaction modeling of combinations of azoles with terbinafine against clinical Scedosporium prolificans isolates. Antimicrob. Agents Chemother. 2003; 47:106-117). Based on the model assumption that a drug does not self-interact, an FIC index is the sum of FICs of each drug tested, where the FIC is determined for each drug by dividing the isoeffective concentration of each drug when used in combination by the MIC of each drug when used alone. Drug interactions were classified as synergistic if the FIC index was ≦0.5, indifferent if >0.5-4, and antagonistic if >4.

Results

Minimal inhibitory concentrations (MICs) of K408 and azoles alone and when combined, and the fractional inhibitory concentration (FIC) indices of the combinations against azole-resistant Candida albicans ATCC 64124.

| | ^{a}MIC (mg L⁻¹) | | | ^{b}FIC Index |
|---|---|---|---|---|
| Antifungal | | | K408 with | (K408 with |
| compound | alone | with K408 | azole | azole) |
| K408 | 31.25, 31.25 | — | — | — |
| chlotrimazole | 3.91, 7.81 | 0.24, 0.49 | 3.91, 1.95 | 0.37, 0.13 |
| fluconazole | 250, 250 | 15.63, 15.63 | 7.81, 7.81 | 0.31, 0.31 |
| itraconazole | 250, 250 | 0.98, 0.98 | 7.81, 7.81 | 0.25, 0.25 |
| posaconazole | 62.50, 250 | 0.24, 0.24 | 3.91, 1.95 | 0.38, 0.07 |
| voriconazole | 7.81, 7.81 | 1.95, 0.81 | 3.91, 0.98 | 0.38, 0.38 |

^{a}Data shown are from two separate experiments
^{b}FIC, fractional inhibitory concentration. Combination interactions are classified as synergistically inhibitory if the FIC index is ≦0.5, indifferent if >0.5-4, and antagonistic if >4.

Minimal inhibitory concentrations (MICs) of K4608 and azoles alone and when combined, and the fractional inhibitory concentration (FIC) indices of the combinations against azole-resistant Candida albicans ATCC 64124.

| | ^{a}MIC (mg L⁻¹) | | | ^{b}FIC Index |
|---|---|---|---|---|
| Antifungal | | with | K4608 with | (K4608 with |
| compound | alone | K4608 | azole | azole) |
| K4608 | 31.25 | — | — | — |
| chlotrimazole | 7.81, 31.25 | 3.91 | 1.95 | 0.19 |
| fluconazole | >250 | 31.25 | 3.91 | 0.25 |
| itraconazole | >250 | 3.91 | 15.63 | <0.52 |
| posaconazole | 62.50, 250 | 0.49 | 3.91 | <0.13 |
| voriconazole | 7.81 | 0.98 | 1.95 | 0.19 |

^{a}Results based on one experiment
^{b}FIC, fractional inhibitory concentration. Combination interactions are classified as synergistically inhibitory if the FIC index is ≦0.5, indifferent if >0.5-4, and antagonistic if >4.

### IV. Cytotoxicity Tests

To test the efficacy of the above prepared compounds, the following procedures were used.

Test Procedures

Cell Culture.

HeLa cells were grown in commercial DMEM 1× (Gibco) with 10% fetal bovine serum (FBS, Gibco), 100 U/mL penicillin, and 100 μg/mL streptomycin at 37° C. and 5% CO₂. The cells were allowed to adhere for 24 h before drug treatment.

Cell Viability Assay.

The cells were seeded in 96-well microtiter plates (10,000/200 μL). After 24 h of incubation in the media, cells were treated with various concentrations of K608, K4608 and K408 (0.0, 1.0, 10.0, 100.0, 1000 μg/mL and Trition 1×) for 48 h. 20 μL of MTT stock solution (5 mg/mL) was added to each well and incubated for 4 h at 37° C. Upon completion of incubation, the media was carefully removed and washed twice with 100 mL of pbs buffer. Then, 200 μL of DMSO was added to each well; agitated on orbital shaker for 15 min., and the absorbance at 570 nm with 670 nm filter was determined with a microplate reader (Synergy H4). The results are expressed as viability compared with that of control. The experiment was carried out in triplicate in three independent experiments.

Results

The results show K608, K4608 and K408 are not toxic to human cervical cancer cell line HeLa. In fact, compound K4608 and K408 promoted growth of cells resulting in higher number of viable cells in wells treated with compound. Compound K608 showed very mild cytotoxicity with ˜80% cell viability at all concentration. IC₅₀ values cannot be calculated since the compounds are not very toxic at the concentrations used for this assay. The results are summarized in the below table:

| | | Viable Cells Percentage | | |
|---|---|---|---|---|
| | | Compared to Control | | |
| Drug Concentration | | K608 | K4608 | K408 |
| 1 | μg/mL | 79.47 | 124.28 | 139.37 |
| 10 | μg/mL | 107.9 | 149.52 | 156.92 |
| 100 | μg/mL | 75.36 | 129.86 | 144.82 |
| 1000 | μg/mL | 86.49 | 122.93 | 138.39 |

| | | | |
|---|---|---|---|
| Trition 1X | 4.08 | 6.05 | 4.69 |

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

In the following part of the present specification, numbered examples are listed which are directed to and which define advantageous embodiments. Said examples and embodiments belong to the present disclosure and description. The embodiments, examples, and features as listed, can separately or in groups, be combined in any manner to form embodiments belonging to the present disclosure.

### Numbered Examples

1. A fungicidal compound comprising:

an aminoglycoside, or salt thereof, having the formula:

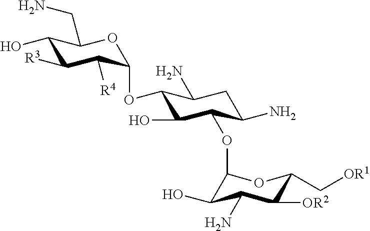

wherein:
R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit,
   wherein n=1 to 10 and R⁵═H or C₁ to C₂₀ branched or straight alkyl;
R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit,
   wherein n=1 to 10 and R⁵═H or C₁ to C₂₀ branched or straight alkyl;
R³ is a member selected from the group consisting of H and OH; and
R⁴ is a member selected from the group consisting of OH and NH₂.

2. The compound of example 1, wherein R¹ and R² are substituted with a group other than H, and the group for R¹ and R² is the same group.

3. The compound of any one of examples 1-2, wherein R¹ and R² comprise a group selected from the group consisting of C(O)OR³ (alkoxycarbonyl), C(O)NHR³ (alkylaminocarbonyl), S(O)₂R³ (alkylsulfonyl), S(O)₂R⁴ (phenylsulfonyl), S(O)R³ (alkylsulfinyl), P(O)₂R³ (alkylphosphonyl), and C(O)R³ (alkanoyl).

4. The compound of any one of examples 1-3, wherein R¹ is H; and R² is a C₆ to C₉ branched or straight alkyl group.

5. The compound of any one of examples 1-4, wherein R¹ is H; and R² is C₈H₁₇.

6. The compound of any one of examples 1-5, wherein R¹ and R² are each a C₆ to C₉ branched or straight alkyl group.

7. The compound any one of examples 1-6, wherein R¹ and R² are each C₆H₁₃.

8. The compound any one of examples 1-6, wherein R¹ and R² are each C₇H₁₅.

9. The compound any one of examples 1-6, wherein R¹ and R² are each C₈H₁₇.

10. A method of treating or preventing a fungal infection which comprises administering to a host in need thereof an effective amount of a fungicidal compound comprising:

an aminoglycoside, or salt thereof, having the formula:

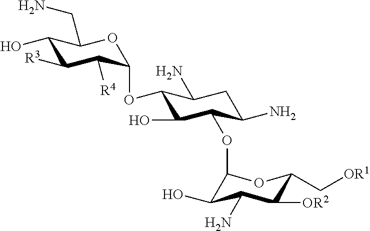

wherein:
R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit,
   wherein n=1 to 10 and R⁵═H or C₁ to C₂₀ branched or straight alkyl;
R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit,
   wherein n=1 to 10 and R⁵═H or C₁ to C₂₀ branched or straight alkyl;
R³ is a member selected from the group consisting of H and OH; and
R⁴ is a member selected from the group consisting of OH and NH₂.

11. The method of example 10, wherein the fungal infection to be treated is caused by an organism selected from the group consisting of Fusarium graminearum, Fusarium oxysporum, Fusarium culmorum, Aspergillus flavus, Aspergillus fumigatus, Aspergillus niger, Altenaria solani, Botrytis cinerea, Botrytis alcata, Phakapsora pachyrhizi, Candida albicans, Candida pseudotropicalis, Candida rugosa, Candida parapsilosis, Candida lusitaniae, Cryptococcus gattis and Cryptococcus neoformans.

12. A fungicidal compound comprising:

an azole; and

an aminoglycoside, or salt thereof, having the formula:

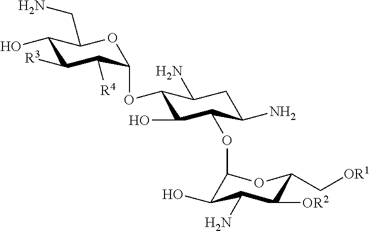

wherein:
   R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit,
      wherein n=1 to 10 and R⁵═H or C₁ to C₂₀ branched or straight alkyl;
   R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit,
      wherein n=1 to 10 and R⁵═H or C₁ to C₂₀ branched or straight alkyl;
   R³ is a member selected from the group consisting of H and OH;
and
   R⁴ is a member selected from the group consisting of OH and NH₂.

13. The compound of example 12, wherein R¹ and R² are substituted with a group other than H, and the group for R¹ and R² is the same group.

14. The compound of any one of examples 12-13, wherein R¹ and R² comprise a group selected from the group consisting of C(O)OR³ (alkoxycarbonyl), C(O)NHR³ (alkylaminocarbonyl), S(O)₂R³ (alkylsulfonyl), S(O)₂R⁴ (phenylsulfonyl), S(O)R³ (alkylsulfinyl), P(O)₂R³ (alkylphosphonyl), and C(O)R³ (alkanoyl).

15. The compound of any one of examples 12-14, wherein R¹ is H; and R² is a C₆ to C₉ branched or straight alkyl group.

16. The compound of any one of examples 12-15, wherein R¹ is H; and R² is C₈H₁₇.

17. The compound of any one of examples 12-16, wherein R¹ and R² are each a C₆ to C₉ branched or straight alkyl group.

18. The compound of any one of examples 12-17, wherein R¹ and R² are selected from the group consisting of C₆H₁₃, C₇H₁₅, and C₈H₁₇.

19. The compound of any one of examples 12-18, wherein the azole is selected from the group consisting of intraconazole, fluconazole, voriconazole, posaconazole, chlotirmazole, tioconazole, ketocaonazole, metconazole, tebuconazole, and pyraclostrobin.

What is claimed is:

1. A fungicidal compound comprising:
an aminoglycoside, or salt thereof, having the formula: 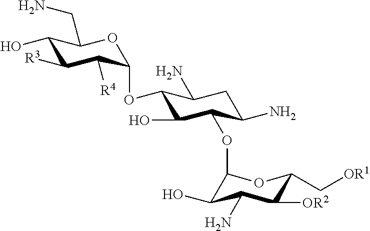
wherein:
R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit,
wherein n=1 to 10 and R⁵═H or C₁ to C₂₀ branched or straight alkyl;
R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit,
wherein n=1 to 10 and R⁵═H or C₁ to C₂₀ branched or straight alkyl;
R³ is a member selected from the group consisting of H and OH; and
R⁴ is a member selected from the group consisting of OH and NH₂.

2. The compound of claim 1, wherein R¹ and R² are substituted with a group other than H, and the group for R¹ and R² is the same group.

3. The compound of claim 1, wherein R¹ and R² comprise a group selected from the group consisting of C(O)OR³ (alkoxycarbonyl), C(O)NHR³ (alkylaminocarbonyl), S(O)₂R³ (alkylsulfonyl), S(O)₂R⁴ (phenylsulfonyl), S(O)R³ (alkylsulfinyl), P(O)₂R³ (alkylphosphonyl), and C(O)R³ (alkanoyl).

4. The compound of claim 1, wherein:
R¹ is H; and
R² is a C₆ to C₉ branched or straight alkyl group.

5. The compound of claim 1, wherein:
R¹ is H; and
R² is C₈H₁₇.

6. The compound of claim 1, wherein R¹ and R² are each a C₆ to C₉ branched or straight alkyl group.

7. The compound of claim 1, wherein R¹ and R² are each C₆H₁₃.

8. The compound of claim 1, wherein R¹ and R² are each C₇H₁₅.

9. The compound of claim 1, wherein R¹ and R² are each C₈H₁₇.

10. A method of treating or preventing a fungal infection which comprises administering to a host in need thereof an effective amount of a fungicidal compound comprising:
an aminoglycoside, or salt thereof, having the formula: 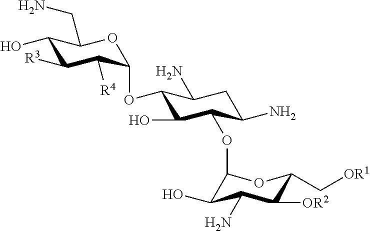
wherein:
R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit,
wherein n=1 to 10 and R⁵═H or C₁ to C₂₀ branched or straight alkyl;
R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit,
wherein n=1 to 10 and R⁵═H or C₁ to C₂₀ branched or straight alkyl;
R³ is a member selected from the group consisting of H and OH; and
R⁴ is a member selected from the group consisting of OH and NH₂.

11. The method of claim 10, wherein the fungal infection to be treated is caused by an organism selected from the group consisting of Fusarium graminearum, Fusarium oxysporum, Fusarium culmorum, Aspergillus flavus, Aspergillus fumigatus, Aspergillus niger, Altenaria solani, Botrytis cinerea, Botrytis alcata, Phakapsora pachyrhizi, Candida albicans, Candida pseudotropicalis, Candida rugosa, Candida parapsilosis, Candida lusitaniae, Cryptococcus gattis and Cryptococcus neoformans.

12. A fungicidal compound comprising:
an azole; and
an aminoglycoside, or salt thereof, having the formula: 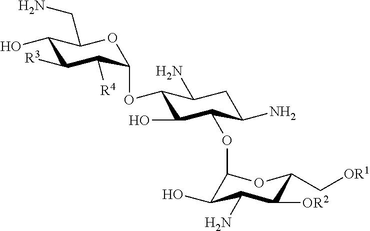
wherein:
R¹ is a member selected from the group consisting of a H; a C₁ to C₂₀ branched or straight alkyl; an unsubstituted phenyl; a substituted phenyl, wherein phenyl group may be C₁ to C₆ alkyl substituted; and a polyethylene glycol —(CH₂CH₂O)ₙR⁵— unit,
wherein n=1 to 10 and R⁵═H or C₁ to C₂₀ branched or straight alkyl;
R² is a member selected from the group consisting of a H; a C₁ to C₂₀ branched or straight alkyl; an unsubstituted phenyl; a substituted phenyl, wherein phenyl group may be C₁ to C₁₀ alkyl substituted; and a polyethylene glycol —(CH₂CH₂O)ₙR⁵— unit,
wherein n=1 to 10 and R⁵═H or C₁ to C₂₀ branched or straight alkyl;
R³ is a member selected from the group consisting of H and OH; and
R⁴ is a member selected from the group consisting of OH and NH₂.

13. The compound of claim 12, wherein R¹ and R² are substituted with a group other than H, and the group for R¹ and R² is the same group.

14. The compound of claim 12, wherein R¹ and R² comprise a group selected from the group consisting of C(O)OR³ (alkoxycarbonyl), C(O)NHR³ (alkylaminocarbonyl), S(O)₂R³ (alkylsulfonyl), S(O)₂R⁴ (phenylsulfonyl), S(O)R³ (alkylsulfinyl), P(O)₂R³ (alkylphosphonyl), and C(O)R³ (alkanoyl).

15. The compound of claim 12, wherein:
R¹ is H; and
R² is a C₆ to C₉ branched or straight alkyl group.

16. The compound of claim 12, wherein:
R¹ is H; and
R² is C₈H₁₇.

17. The compound of claim 12, wherein R¹ and R² are each a C₆ to C₉ branched or straight alkyl group.

18. The compound of claim 12, wherein R¹ and R² are selected from the group consisting of C₆H₁₃, C₇H₁₅, and C₈H₁₇.

19. The compound according to claim 12, wherein the azole is selected from the group consisting of intraconazole, fluconazole, voriconazole, posaconazole, chlotirmazole, tioconazole, ketocaonazole, metconazole, tebuconazole, and pyraclostrobin.
